(19) United States
(12) Patent Application Publication
(10) Pub. No.: US 2022/0135962 A1
(43) Pub. Date: May 5, 2022

(54) METHODS AND MATERIALS FOR BIOLOGICAL IMMOBILIZATION IN MICROFLUIDICS

(71) Applicant: RAN Biotechnologies, Inc, Beverly, MA (US)

(72) Inventors: Roger A. Nassar, Marblehead, MA (US); Robert E. Lintner, Amesbury, MA (US)

(73) Assignee: RAN Biotechnologies, Inc., Beverly, MA (US)

(21) Appl. No.: 17/435,345

(22) PCT Filed: Apr. 17, 2020
(86) PCT No.: PCT/US2020/028776

§ 371 Date: Aug. 31, 2021

Related U.S. Application Data
(60) Provisional application No. 62/812,885, filed on Mar. 1, 2019.

(51) Int. Cl. C12N 11/04; C12N 11/087

(52) U.S. Cl. CPC C12N 11/04; C12N 11/087

(57) ABSTRACT

The present invention is directed to synthesizing and using fluid-insoluble material complexes that capture biologicals and remove them from samples in microscopic scale fluids, such as in droplets, wells, and micro-wells. The present invention also pertains to the option of detecting the captured biologicals, to the option of modifying the captured biologicals, and to the option of controllably releasing the captured biologicals.

### PRIOR APPLICATIONS

This application claims the benefit of and priority to U.S. Provisional Application No. 62/812,885, filed on Mar. 1, 2019, the entire teachings of which are incorporated herein by reference.

### FIELD

The teachings herein relate to methods and materials for purification of biologicals, and more particularly to methods and materials for capturing and immobilizing biologicals on fluid-insoluble material complexes in microfluidic setups.

### BACKGROUND

The use of adsorption chromatography, which includes affinity ligand-matrix conjugates, for purification of biologicals is well established. For example, the use of phenyl-based adsorption chromatography for protein purification, including purification of the in-demand monoclonal antibodies, has been disclosed in the patent literature. Other known adsorption chromatography processes are applied to purification of viruses such as influenza A. However, these processes are generally not designed for microfluidic setups.

Accordingly, there remains a critical need for purification of biological materials in microfluidic setups.

### SUMMARY

The present invention is directed to methods and materials for immobilizing biologicals using fluid-insoluble material complexes that specifically capture microorganisms, microorganism products, proteins, nucleic acids, peptides, and other biologicals within small volumes of fluids on the order of micro-, nano-, pico-liter, or even smaller. It also pertains to the option of controllably releasing the captured biologicals under certain conditions.

In one aspect, a method of immobilizing biologicals is discussed, which includes mixing a sample containing biologicals with material complexes, followed by generating an emulsion of small-volume droplet or droplets which contain the complexed biologicals, and which are suspended in a continuous phase that is immiscible with the phase of the droplets. The material complexes can include hydroxyl-, amino-, mercapto- or epoxy-containing materials that are fluid-insoluble and at least one receptor bound to the materials. The biologicals can include for example any of a cell, tissue, tissue product, blood, blood product, protein, nucleic acids, vaccine, antigen, antitoxin, virus, microorganism, fungus, yeast, alga, and bacterium. If desired, the immobilized biologicals can then be extracted from the material complex, such as by elution. In the case of the virus, the extracted biological can then be included in a vaccine treatment. In the case of the protein, the extracted biological can then be included in a vaccine or therapeutic treatment.

In another aspect, a method of immobilizing biologicals is disclosed, which includes generating two separate emulsions followed by mixing them: the first emulsion is made from small-volume droplet or droplets which contain the biologicals, and which are suspended in a continuous phase that is immiscible with the phase of the droplets; and the second emulsion is made from small-volume droplet or droplets which contain the material complexes and which are suspended in a continuous phase that is immiscible with the phase of the droplets. The two emulsions are then mixed allowing the controlled or un-controlled fusion of two or more droplets from these two emulsions, where at least one droplet from each emulsion is represented. The new fused droplets can simultaneously contain biologicals and material complexes, allowing for immobilization of the biologicals on the material complexes. The material complexes can include hydroxyl-, amino-, mercapto- or epoxy-containing materials that are fluid-insoluble and at least one receptor bound to the materials. The biologicals can include, for example, any of a cell, tissue, tissue product, blood, blood product, protein, nucleic acids, vaccine, antigen, antitoxin, virus, microorganism, fungus, yeast, alga, and bacterium. If desired, the immobilized biologicals can then be extracted from the material complex, such as by elution. In the case of the virus, the extracted biological can then be included in a vaccine treatment. In the case of the protein, the extracted biological can then be included in a vaccine or therapeutic treatment.

In yet another aspect, a method according to the present teachings can include mixing the starting materials of material complexes with biologicals, followed by generating an emulsion of small-volume droplet or droplets which contain the starting materials and the biologicals, and which are suspended in a continuous phase that is immiscible with the phase of the droplets. The next step is allowing the in-situ formation of material complexes, while simultaneously immobilizing biologicals on the material complexes. The material complexes can include hydroxyl-, amino-, mercapto- or epoxy-containing materials, hydrogels, poly-lactic-containing polymers, that are fluid-insoluble and at least one receptor bound to the materials. The biologicals can include for example any of a cell, tissue, tissue product, blood, blood product, protein, nucleic acids, vaccine, antigen, antitoxin, virus, microorganism, fungus, yeast, alga, and bacterium. If desired, the immobilized biologicals can then be extracted from the material complex, such as by elution. In the case of the virus, the extracted biological can then be included in a vaccine treatment. In the case of the protein, the extracted biological can then be included in a vaccine or therapeutic treatment.

In yet another aspect, methods and materials for forming the aforementioned materials, material complexes, mixtures, compositions, composites, emulsions, or any combination thereof, and for purifying, immobilizing, capturing, and separating the aforementioned biologicals in wells and micro-wells instead of droplets are disclosed.

In another aspect, a method for immobilizing a biological is disclosed, which includes mixing a fluid sample comprising the biological with a material complex comprising a hydroxyl-, amino-, mercapto or epoxy-containing material that is fluid-insoluble and at least one receptor selected from lactose, lactose derivative, mono- or poly-saccharide, heparin, chitosan, deoxyribonucleic acid, ribonucleic acid, peptide, photoreceptor, or any combination thereof. The receptor can be bound to the material. The method can also include suspending the fluid sample in at least one immiscible fluid and separating the biological from the fluid sample by adsorbing the biological to the material complex.

In some aspects, the biological can be selected from the group consisting of cell, cell product, tissue, tissue product, blood, blood product, body fluid, product of body fluid, protein, nucleic acid, vaccine, antigen, antitoxin, biological medicine, biological treatment, virus, virus product, microorganism, microorganism product, fungus, yeast, alga, bacterium, prokaryote, eukaryote, Staphylococcus aureus, Streptococcus, Escherichia coli (E. coli), Pseudomonas aeruginosa, mycobacterium, adenovirus, rhinovirus, smallpox virus, influenza virus, herpes virus, human immunodeficiency virus (HIV), rabies, chikungunya, severe acute respiratory syndrome (SARS), polio, malaria, dengue fever, tuberculosis, meningitis, typhoid fever, yellow fever, ebola, shingella, listeria, yersinia, West Nile virus, protozoa, fungi Salmonella enterica, Candida albicans, Trichophyton mentagrophytes, poliovirus, Enterobacter aerogenes, Salmonella typhi, Klebsiella pneumonia, Aspergillus brasiliensis, methicillin resistant Staphylococcus aureus (MRSA), any derivative thereof, or any combination thereof.

In some aspects, the material can be selected from the group consisting of agarose, sand, textiles, metallic particles (including nanoparticles), magnetic particles (including nanoparticles), glass, fiberglass, silica, wood, fiber, plastic, rubber, ceramic, percelain, stone, marble, cement, biological polymers, natural polymers, synthetic polymers, poly acrylamide polymers, poly lactic polymers, gel, colloidal gel, hydrogel, any derivative thereof, or any combination thereof.

In some aspects, the receptor can be bound directly to the material. In other aspects, the receptor can be bound indirectly to the material, e.g., via a linker.

In some aspects, the linker can be selected from the group consisting of linear poly(ethylene glycol) (PEG), branched PEG, linear poly(ethylenimine) (PEI, various ratios of primary:secondary:tertiary amine groups), branched PEI, a dendron, a dendrimer, a hyperbranched bis-MPA polyester-16-hydroxyl, chitosan, any derivative thereof, or any combination thereof.

In some aspects, the inter-bonding between any combination of receptor, material, and the linker can be achieved using at least one chemical coupling reagent. In these aspects and in other aspects, the coupling reagent can be selected from the group consisting of 1,1′-carbonyldiimidazole (CDI), N,N′-Dicyclohexylcarbodiimide (DCC), N-(3-Dimethylaminopropyl)-N′-ethylcarbodiimide hydrochloride (EDC or EDCI), or any combination thereof.

In some aspects, the inter-bonding between any combination of receptor, material, and the linker can be achieved using physical attachment, chemical attachment, or a combination of chemical and physical attachments. In these aspects and other aspects, the physical attachment can be achieved by deposition of the receptor, the linker, or a combination thereof, onto the material in a controlled fashion, a non-controlled fashion, or a combination thereof.

In some aspects, the material can be chemically functional and the chemical functionality can be amino, ammonium, hydroxyl, mercapto, sulfone, sulfinic acid, sulfonic acid, thiocyanate, thione, thial, thiol, carboxyl, halocarboxy, halo, imido, anhydrido, alkenyl, alkynyl, phenyl, benzyl, carbonyl, formyl, haloformyl, carbonato, ester, alkoxy, phenoxy, hydroperoxy, peroxy, ether, glycidyl, epoxy, hemiacetal, hemiketal, acetal, ketal, orthoester, orthocarbonate ester, amido, imino, imido, azido, azo, cyano, nitrato, nitrilo, nitrito, nitro, nitroso, pyridinyl, phosphinyl, phosphonic acid, phosphate, phosphoester, phosphodiester, boronic acid, boronic ester, borinic acid, borinic ester, any derivative thereof, or any combination thereof. In these aspects and other aspects, the epoxy-containing material can be Poly(glycidyl methacrylate) (PGMA) and the amino-containing material can be PGMA-NH₂.

In some aspects, the hydroxyl, mercapto, or amino group can be formed on a surface of the material by modifying the substrate by a chemical transformation. In these aspects, the chemical transformation can comprise a hydrolysis reaction with an acid, a base, or a combination thereof.

In some aspects, the material complex can be formed within the fluid sample and the biological can be encapsulated or immoblized in or on the material complex.

In some aspects, a method for immobilizing a biological is disclosed, which includes separating an immobilized biological from a fluid sample by filtration, decantation, applying gravity or magnetic forces, flow cytometry, fluorescence-activated cell sorter, or any combination thereof. In these aspects and other aspects, the method can include releasing the immobilized biological from the material complex by, for example, light-inducing variations, enzymatic activity, physical variations, chemical variations, or any combination thereof. In these aspects and other aspects, the method can include releasing the immobilized biological from the material complexby, for example, temperature variations, irradiation variations, mechanical variations, thermodynamic variations, thermomechanic variations, or any combination thereof. In these aspects and other aspects, the method can include releasing the immobilized biological from the material complexby, for example, variations in pH values, concentration of chemicals, concentration of ions, concentration of sodium chloride, or any combination thereof.

In some aspects, a method for immobilizing a biological is disclosed, wherein the method can be part of a process, production, operation, kit, or application of medicine, vaccine, medical device, diagnostic equipment and techniques, implant, glove, mask, textile, surgical drape, tubing, surgical instrument, safety gear, fabric, apparel item, floor, handle, wall, sink, shower, tub, toilet, furniture, wall switch, toy, athletic equipment, playground equipment, shopping cart, countertop, appliance, railing, door, air filter, air processing equipment, water filter, water processing equipment, pipe, phone, cell phone, remote control, computer, mouse, keyboard, touch screen, leather, cosmetic, cosmetic making equipment, cosmetic storage equipment, personal care item, personal care item making equipment, personal care storage equipment, animal care item, animal care item making equipment, animal care storage equipment, veterinary equipment, powder, cream, gel, salve, eye care item, eye care item making equipment, eye care storage equipment, contact lens, contact lens case, glasses, jewelry, jewelry making equipment, jewelry storage equipment, utensil, dish, cup, container, object display container, food display container, food package, food processing equipment, food handling equipment, food transportation equipment, food storage equipment, food vending equipment, animal housing, farming equipment, animal food handling equipment, animal food storage space, animal food processing equipment, animal food storage equipment, animal food container, air vehicle, land vehicle, water vehicle, water storage space, water storage equipment, water storage container, water processing equipment, water storage equipment, water filter, air filter, or any combination thereof.

In another aspect, a method for protecting an object against microbial infection, microbial colonization, or microbial trans-infection is disclosed, which includes providing to the object a microbial barrier according to one or more of the methods disclosed herein.

In some aspects, a method for immobilizing a biological is disclosed, which include detecting the immobilized biological, modifying the immobilized biological, or detecting and modifying the immobilized biological. In these aspects and other aspects, the modified immobilized biological can be released from the material complex according to one or more methods disclosed herein.

In some aspects, the immiscible fluid can be in a well.

In another aspect, a material complex is disclosed, which includes a hydroxyl-, amino-, mercapto or epoxy-containing material and at least one receptor bound to the material and selected from lactose, lactose derivative, mono- or poly-saccharide, heparin, chitosan, deoxyribonucleic acid, ribonucleic acid, peptide, photoreceptor, or any combination thereof. In this aspect and other aspects, the material complex can be dispersed in a second fluid. In this aspect and other aspects, the first fluid can be suspended in an immiscible second fluid.

These and other aspects of the applicants' teaching are set forth herein.

### BRIEF DESCRIPTION OF THE DRAWINGS

The foregoing and other objects and advantages of the invention will be appreciated more fully from the following further description, with reference to the accompanying drawings. The skilled person in the art will understand that the drawings, described below, are for illustration purposes only. The drawings are not intended to limit the scope of the applicants' teachings in any way.

FIG. 1 schematically illustrates three different embodiments of fluid-insoluble material cores that are complexed with receptors either directly or indirectly through linkers in accordance with various aspects of the applicants' teachings;

FIG. 2 schematically illustrates examples of direct attachment of receptors to materials in accordance with various aspects of the applicants' teachings;

FIGS. 3A, 3B, and 3C schematically illustrate examples of attachment of receptors to materials via linkers in accordance with various aspects of the applicants' teachings;

FIG. 4 schematically illustrates a general route for covalent coupling when using 1,1′-carbonyldiimidazole in accordance with various aspects of the applicants' teachings;

FIG. 5 schematically illustrates the emulsification of biologicals immobilized on fluid-insoluble material complexes in accordance with various aspects of the applicants' teachings;

FIG. 6 schematically illustrates the fusion of two emulsions, Emulsion A made from droplets containing fluid-insoluble material complexes and Emulsion B made from droplets containing biologicals, in accordance with various aspects of the applicants' teachings;

FIG. 7 schematically illustrates engineered emulsification of homogeneously-sized droplets, each containing biologicals immobilized on fluid-insoluble material complexes, using a microfluidic chip in accordance with various aspects of the applicants' teachings;

FIG. 8 schematically illustrates the fusion of two engineered emulsions of homogeneously-sized droplets, the first set of droplets contains fluid-insoluble material complexes and the second set of droplets contains biologicals, in accordance with various aspects of the applicants' teachings;

FIG. 9 is a diagram of the chemical derivatization of materials monitored by recombinant HA binding assays in accordance with various aspects of the applicants' teachings;

FIG. 10 is a diagram of the concentration of the captured virus in accordance with various aspects of the applicants' teachings; and

FIG. 11 is a diagram of the adsorbed virus and initial virus in accordance with various aspects of the applicants' teachings.

### DETAILED DESCRIPTION OF THE INVENTION

The present invention is directed to the methods and materials for capturing and immobilizing biologicals on fluid-insoluble material complexes in microfluidic setups. It also pertains to the option of controllably releasing the captured biologicals under specific conditions.

Certain exemplary embodiments will now be described to provide an overall understanding of the principles of the structure, function, manufacture, and use of the methods and materials disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

All publications, patents, and patent applications cited herein, whether supra or infra, are hereby incorporated by reference in their entirety. As used in this specification and the appended claims, the singular forms “a,” “an,” and “the” include plural references unless the content clearly dictates otherwise. The terms used in this invention adhere to standard definitions generally accepted by those having ordinary skill in the art. In case any further explanation might be needed, some terms have been further elucidated below.

The term “biologicals” as used herein refers to living organisms and their products, including, but not limited to, cell, tissue, tissue product, blood, blood product, protein, deoxyribonucleic acid, ribonucleic acid, nucleic acid, vaccine, antigen, antitoxin, viruses, microorganism, fungi, yeast, algae, bacteria, derivative thereof, or any combination thereof. One example of biological can include microorganism, such as pathogenic or non-pathogenic bacteria. Other examples of biologicals can include viruses, viral products, virus-imitating entities, derivative thereof, or any combination thereof.

The term “about” as used herein denotes a variation of at most 10% around a numerical value.

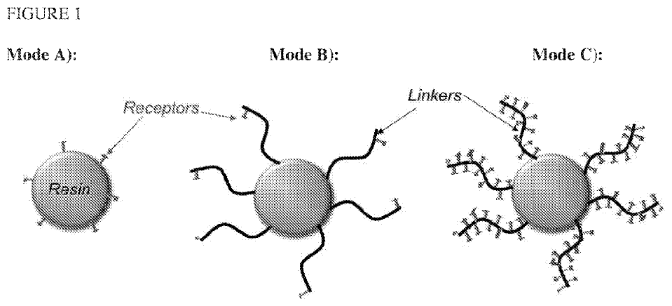

In one embodiment, fluid-insoluble materials can be complexed with microoganism-capturing groups (also called “receptors”), the structures of which are drawn from natural cellular receptors, antibodies, or simply from available data describing microoganism interaction with soluble molecules. The receptors can be directly attached to the material (FIG. 1, Mode A) or through a linker (FIG. 1, Mode B). In order to protect the integrity of the molecular structure of the subject material complexes, particularly when re-cycling is a requirement, one method of inter-connecting the receptors, linkers and materials can be via covalent bonding. For certain applications where added structural stability is not needed, for example in single use material complexes, physical bonding can substitute covalent bonding. The receptors play a direct role by capturing the microorganims through physical bonding, e.g., by hydrogen bonding. One role of linkers is to position the receptors at an active distance from the core of the material. By distancing the receptors from the core of the material, the receptors can easily access the target microorganisms. Another role for the linkers, particularly when they are branched, is to increase the density of the receptors on the surface of the material (FIG. 1, Mode C). In many embodiments, an increase in the density of receptors correlates with an increase in the capacity of capturing higher concentrations of microorganisms.

Examples of the three main components of the material complexes are: 1) materials: agarose, sand, textiles (e.g., cellulose/cotton, wool, nylon, polyester), metallic particles (e.g., nanoparticles), magnetic particles (e.g., nanoparticles), glass, fiberglass, silica, wood, fiber, plastic, rubber, ceramic, percelain, stone, marble, cement, biological polymers, natural polymers and synthetic polymers (e.g., PGMA), derivative thereof, or any combination thereof; 2) receptors: lactose (natural and synthetic) and its derivatives (e.g., sialyllactose), mono- and poly-saccharides (natural and synthetic), heparin and chitosan, derivative thereof, or any combination thereof; and 3) linkers: linear and branched polymers, such as poly(ethylene glycol) (PEG) and poly(ethylenimine) (PEI, various ratios of primary:secondary:tertiary amine groups), (e.g., multi-arm branched PEG-amines), dendrons and dendrimers (e.g., hyperbranched bis-MPA polyester-16-hydroxyl), chitosan, derivative thereof, or any combination thereof. Each of the material complexes may incorporate the material and the receptor components. However, incorporating the linker component is optional.

Metal materials suitable for use in the invention include, for example, stainless steel, nickel, titanium, tantalum, aluminum, copper, gold, silver, platinum, zinc, Nitinol, Inconel, iridium, tungsten, silicon, magnesium, tin, alloys, coatings containing any of the foregoing, galvanized steel, hot dipped galvanized steel, electrogalvanized steel, annealed hot dipped galvanized steel, derivative thereof, or any combination thereof.

Glass materials suitable for use in the invention include, for example, soda lime glass, strontium glass, borosilicate glass, barium glass, glass-ceramics containing lanthanum, derivative thereof, or any combination thereof.

Sand materials suitable for use in the invention include, for example, sand comprising silica (e.g., quartz, fused quartz, crystalline silica, fumed silica, silica gel, and silica aerogel), calcium carbonate (e.g., aragonite), derivative thereof, or any combination thereof. The sand can comprise other components, such as minerals (e.g., magnetite, chlorite, glauconite, gypsum, olivine, garnet), metal (e.g., iron), shells, coral, limestone, rock, derivative thereof, or any combination thereof.

Wood materials suitable for the invention include, for example, hard wood, soft wood, and materials engineered from wood, wood chips, and fiber (e.g., plywood, oriented strand board, laminated veneer lumber, composites, strand lumber, chipboard, hardboard, and medium density fiberboard), derivative thereof, or any combination thereof. Types of wood include alder, birch, elm, maple, willow, walnut, cherry, oak, hickory, poplar, pine, fir, or any combination thereof.

Fiber materials suitable for use in the invention include, for example, natural fibers (e.g., derived from an animal, vegetable, or mineral) and synthetic fibers (e.g., derived from cellulose, mineral, or polymer). Suitable natural fibers include, for example, cotton, hemp, jute, flax, ramie, sisal, bagasse, wood fiber, silkworm silk, spider silk, sinew, catgut, wool, sea silk, wool, mohair, angora, and asbestos. Suitable synthetic fibers include, for example, rayon, modal, Lyocell, metal fiber (e.g., copper, gold, silver, nickel, aluminum, iron), carbon fiber, silicon carbide fiber, bamboo fiber, seacell, nylon, polyester, polyvinyl chloride fiber (e.g., vinyon), polyolefin fiber (e.g., polyethylene, polypropylene), acrylic polyester fiber, aramid, spandex, or any combination thereof.

Natural polymer materials suitable for use in the invention include, for example, a polysaccharide (e.g., cotton, cellulose), shellac, amber, wool, silk, natural rubber, and a biopolymer (e.g., a protein, an extracellular matrix component, collagen), or any combination thereof.

Synthetic polymer materials suitable for use in the invention include, for example, polyvinylpyrrolidone, acrylics, acrylonitrile-butadiene-styrene, poly acrylonitrile, acetals, polyphenylene oxides, polyimides, polystyrene, polypropylene, polyethylene, polytetrafluoroethylene, polyvinylidene fluoride, polyvinyl chloride, polyethylenimine, polyesters, polyethers, polyamide, polyorthoester, polyanhydride, polysulfone, polyether sulfone, polycaprolactone, polyhydroxy-butyrate valerate, polylactones, polyurethanes, polycarbonates, polyethylene terephthalate, copolymers, derivative thereof, or any combination thereof.

Typical rubber materials suitable for use in the invention include, for example, silicones, fluorosilicones, nitrile rubbers, silicone rubbers, polyisoprenes, sulfur-cured rubbers, butadiene-acrylonitrile rubbers, isoprene-acrylonitrile rubbers, derivative thereof, or any combination thereof.

Ceramic materials suitable for use in the invention include, for example, boron nitrides, silicon nitrides, aluminas, silicas, the like, derivative thereof, or any combination thereof.

Stone materials suitable for use in the invention include, for example, granite, quartz, quartzite, limestone, dolostone, sandstone, marble, soapstone, serpentine, derivative thereof, and any combination thereof.

Exemplary receptors can include: 1) heparin, a negatively charged polymer that can mimic innate glycosaminoglycanes found in the memebranes of host cells. It is commercially available as heparin sodium which is extracted from porcine intestinal mucosa and is approved as blood anti-coagulant. Also, non-animal-derived synthetic heparin-mimicking sulfonic acid polymers can act in a similar fashion to natural heparin; 2) chitosan, an ecologically friendly bio-pesticide that can ligate to a variety of microorganisms and proteins. It is also used as a hemostatic agent and in transdermal drug delivery; and 3) lactose, a by-product of the dairy industry. It is widely available and produced annually in millions of tons. Lactose can also be synthesized by condensation/dehydration of the two sugars, galactose and glucose, including all their isomers. Exemplary receptors can also include heparin derivative, chitosan derivative, lactose derivative, or any combination thereof.

Exemplary materials can include: 1) sand, an affordable and widely available material. In addition, complexed sand could easily replace non-complexed sand in established technologies such as drinking water purification; 2) agarose, particularly Sepharose®, a beaded polysaccharide polymer extracted from seaweed. They are also widely available and used in chromatography to separate biomolecules; and 3) PGMA, a synthetic polymer produced from Glycidyl methacrylate, which is an ester of methacrylic acid and a common monomer used in the production of epoxy materials.

Exemplary linkers can include: 1) chitosan (see its description as a receptor); 2) poly(ethylene glycol)(PEG) and its derivatives, produced from ethylene oxides with many different chemical, biological, commercial, and industrial uses; and 3) dendrons and dendrimers, relatively new molecules. They are repetitively branched molecules using a small number of starting reagents. They are commonly used in drug delivery and sensors. Some suitable examples of dendrons and dendrimers include, without limitation, hydroxyl-terminated polyester dendrons, amine-terminated carbosilane dendrons, and hydroxyl-terminated polyether dendrons.

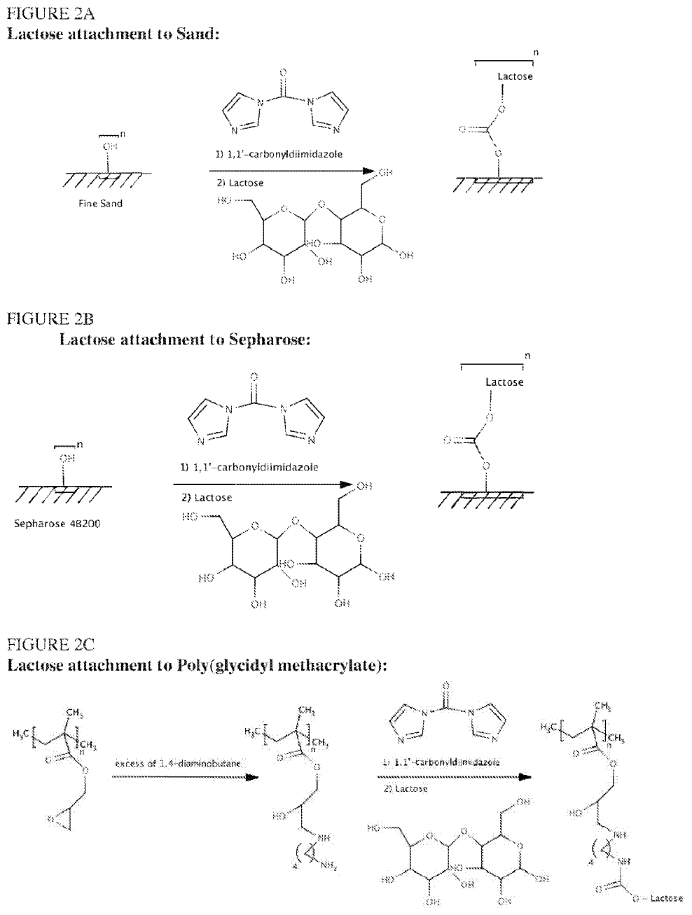

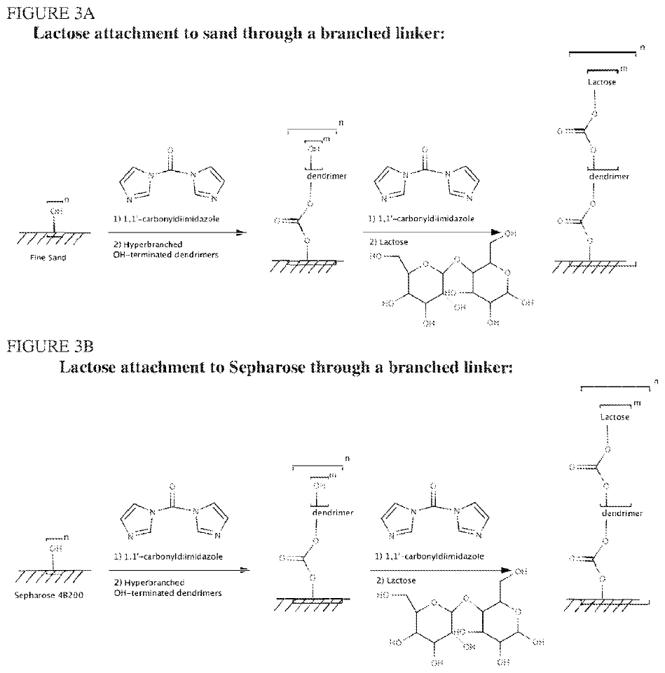

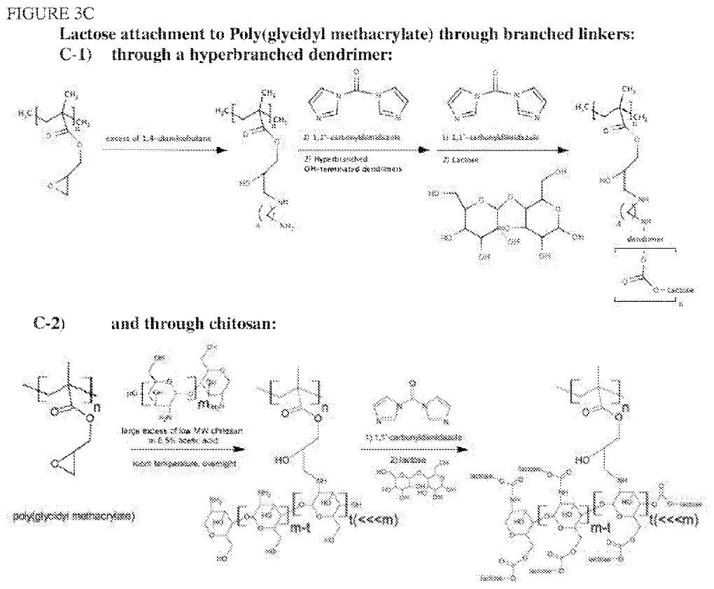

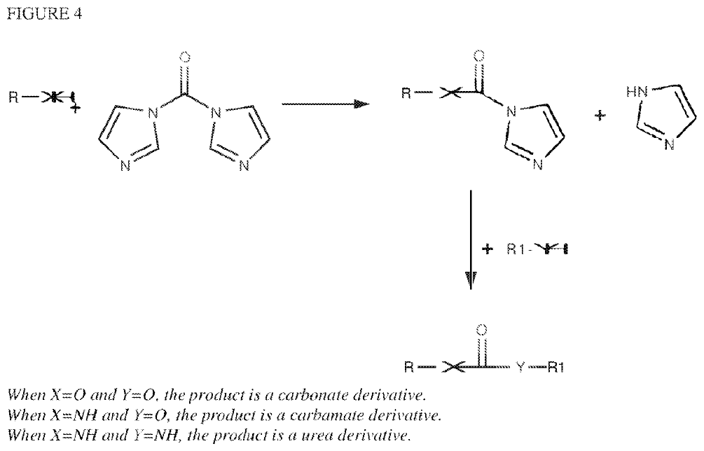

In one aspect, the receptors can be directly attached to the material (FIG. 2) or through linkers (FIG. 3) via chemical coupling. One type of coupling reagent is 1,1′-carbonyldiimidazole (CDI). The coupling reagent may also be N,N′-Dicyclohexylcarbodiinide (DCC) or N-(3-Dimethylaininopropyl)-N′-ethylcarbodiimide hydrochloride (EDC or EDCT).

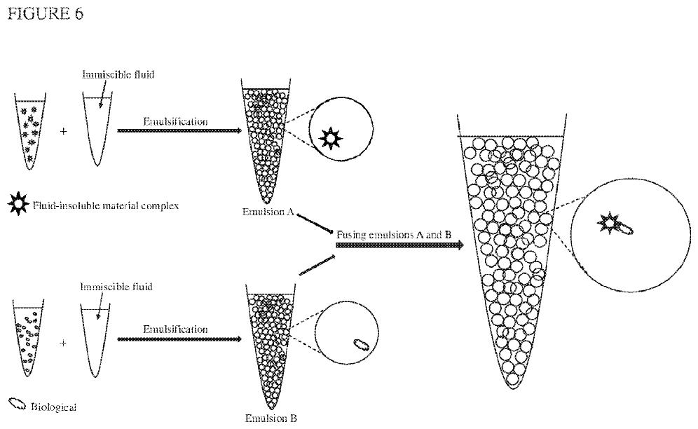

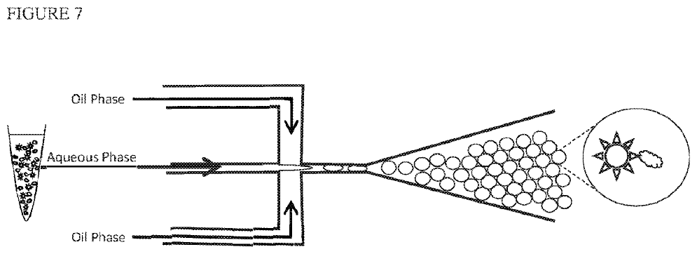

An exemplary coupling reagent is CDI. Basic protonated end groups, such as hydroxyl groups (R—OH) in sand and Sepharose® and tertiary amine groups (R—NH₂) in PGMA-diaminobutane, readily react with CDI to form an ester or amide link. The resulting imidazole-substituted derivatives are reacted with hydroxyl-terminated receptors yielding either carbonates [R—O—C(O)—O-receptor] or carbamates [R—N(H)—C(O)—O-receptor]. The resulting imidazole-substituted derivatives can also be reacted with amine-terminated receptors yielding urea derivatives [R—N(H)—C(O)—N(H)-receptor] (FIG. 4). Due to the formation of a covalent bound between the receptor and the material (via direct bonding or through a linker), the structure of the bound receptor is different compared to the structure of the commercially available free receptor. For example, as depicted in FIG. 6, the receptor can lose a hydrogen atom upon reaction with the immidazole-substituted derivatives to form a receptor-carbonate, receptor-carbamate, or receptor-urea derivative.

When an appropriate functional group is not present on the surface of the material, a suitable functional group can be made available to the surface by a chemical transformation. In general, a chemical transformation can be hydrolysis, oxidation (e.g., using Collins reagent, Dess-Martin periodinane, Jones reagent, and potassium permanganate), reduction (e.g., using sodium borohydride or lithium aluminum hydride), alkylation, deprotonation, electrophilic addition (e.g., halogenation, hydrohalogenation, and hydration), hydrogenation, esterification, elimination reaction (e.g., dehydration), nucleophilic substitution, radical substitution, or a rearrangement reaction. If needed, more than one chemical transformation, successively or simultaneously, can be used to provide a suitable functional group or a heterogeneous group of functional groups of various identities. Alternatively, a monomer with a desired functional group can be grafted to the material.

In some embodiments, the chemical transformation is hydrolysis. Generally, hydrolysis is performed with water in the presence of a strong inorganic, organic, or organo-metallic acid (e.g., strong inorganic acid, such as hydrochloric acid, sulfuric acid, phosphoric acid, nitric acid, hydroiodic acid, hydrobromic acid, chloric acid, and perchloric acid) or strong inorganic, organic, or organo-metallic base (e.g., Group I and Group II hydroxides, such as lithium hydroxide, sodium hydroxide, potassium hydroxide, rubidium hydroxide, cesium hydroxide, magnesium hydroxide, calcium hydroxide, and barium hydroxide; ammonium hydroxide; and sodium carbonate). For example, a material comprising an acyl halide can undergo hydrolysis to form a carboxylic acid.

In some embodiments, the chemical transformation is a substitution reaction where one functional group is replaced with another. For example, a material comprising a haloalkyl group can react with a strong base to form a hydroxy group.

In other aspects, the chemical transformation is alkylation, hydrogenation, or reduction. For example, a material comprising a hydroxy or haloalkyl (e.g., iodoalkyl or bromoalkyl) moiety can be reacted with ammonia to form an amino group. A material comprising a haloalkyl moiety also can be converted to a mercapto group by S-alkylation using thiourea. A material comprising a nitrile can be hydrogenated to form an amino group. A material comprising an amido group can be reduced (e.g., in the presence of lithium aluminum hydride) to form an amino group. A material comprising a formyl or keto group can be reduced to form an amino or hydroxy group. Multiple homogeneous or heterogeneous transformations can be applied simultaneously or successively.

A variety of material complexes can be used in the present invention such as the ones disclosed in U.S. Pat. No. 10,105,681 and US Pub. No. 2016/0010136 which are herein incorporated by reference in their entirety. Material complexes comprise, for example, lactose-Sepharose, lactose-sand, lactose-PGMA, heparin-Sepharose, heparin-sand, heparin-PGMA, lactose-[branching]-Sepharose, lactose-[branching]-sand, lactose-[branching]-PGMA, heparin-[branching]-Sepharose, heparin-[branching]-sand, heparin-[branching]-PGMA, and derivatives thereof. The material complexes can be formed by any suitable method using suitable temperatures (e.g., room temperature and reflux), reaction times, solvents, catalysts, and concentrations. In some aspects, an excess amount of linkers and receptors can be used to ensure an effective amount of receptors in the material complexes.

In another aspect, attachments amongst receptors, linkers, and materials can be secured physically. This is achieved by mixing receptors or linkers, or any combination thereof, dissolved in one or more solvents with the materials, then allowing the one or more solvents to evaporate in air, under vacuum, or a combination thereof.

The receptors may also reversibly interact with the target biologicals, such as micro-organisms or viruses. The biologicals can be desorbed from the receptors, such as through elution. Eluents such as higher-than-physiological sodium chloride solutions and lactose-containing solutions are capable of desorbing the biologicals from the material complexes.

Depicted in FIGS. 2 and 3, one exemplary receptor is lactose. Immobilized lactose can be used for capturing a high titer of influenza A virus. Furthermore, lactose-PGMA combination is also an exemplary material.

The material complexes can be used for the capture of biologicals in fluids. These material complexes should not dissolve in the aforementioned fluids.

The disclosed methods and material complexes may be used in a number of applications including, for example: 1) pharmaceuticals: culturing microorganisms, inoculating microorganisms, purification of vaccines, proteins, including monoclonal antibodies (MAbs), and other biologicals; 2) diagnostics: increasing the concentration of target biologicals in samples leading to increase in sensitivity in existing and novel diagnostic tools, or including materials that change color upon binding a biological molecule or exhibit a signal indicating their binding to biologicals and allowing simple point-of-use diagnostics; 3) prophylactics: trapping biologicals prior to infection or contamination (e.g. face masks, air purifiers, and gloves); 4) therapeutics: disinfection of blood and its products, extracorporeal dialysis, disinfection of intestinal fluids, and controlling the biological composition of life-sustaining fluids; and 5) environmental: removing biologicals from water and other fluids in the environment, including air.

In one embodiment, the disclosed methods and material complexes can be used for vaccine purification. Current vaccine purification techniques use a combination of membrane separation (e.g., ultrafiltration) and chromatographic separation (e.g., size exclusion and ion exchange). While the overall purity is above about 90%, the yield is only about 50%. The disclosed methods and material complexes can substitute the separations based on size exclusion, ion exchange chromatography, or a combination thereof. When the disclosed methods and material complexes show high selectivity towards target biologicals, it is possible that the disclosed methods and material complexes could substitute chromatograpic separations, membrane separation, other filtration steps, or any combination thereof.

In another embodiment, the disclosed methods and material complexes can be used in microfluidic setups. Such setups have the advantage of allowing the execution and study of reactions and interactions on very small microscopic scale, which leads to amplified signals and minimized noises due to irrelevant reactions and interactions.

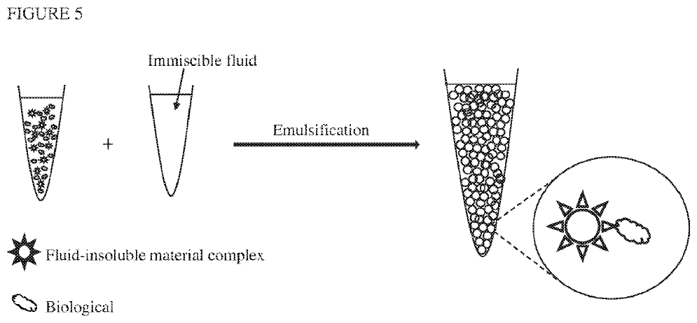

In another embodiment, the disclosed methods and material complexes combined with target biologicals can be combined with a non-miscible fluid (FIG. 5). The mixture can then be emulsified via shaking, vortexing, other technical emulsification procedures, or any combination thereof. The resulting emulsion can be composed of droplets suspended in the non-miscible fluid. Each droplet can contain material complexes, target biologicals, or a combination thereof.

Non-miscible fluids suitable for use in the invention include, for example, mineral oils, hydrocarbon oils, vegetable oils, parafin oils, fluorinated oils, fully fluorinated oils, partially fluorinated oils, any derivative thereof, or any combination thereof.

In another embodiment, the disclosed methods and material complexes can be combined with a non-miscible fluid to form Emulsion A; and the disclosed methods and biologicals can be combined with a non-miscible fluid to form Emulsion B (FIG. 6). The emulsifications can be achieved via shaking, vortexing, other technical emulsification procedures, or any combination thereof. The two resulting emulsions, A and B, can be combined and droplets can be controllably or un-controllably merged, facilitating potential interactions between material complexes and biologicals.

In another embodiment, the disclosed methods and material complexes combined with target biologicals can be combined with a non-miscible fluid in a controlled or engineered method to form an engineered emulsion (FIG. 7). An example of controlled or engineered method is by using a microfluidic chip. The resulting emulsion is a mixture of droplets containing material complexes, target biologicals, or a combination thereof.

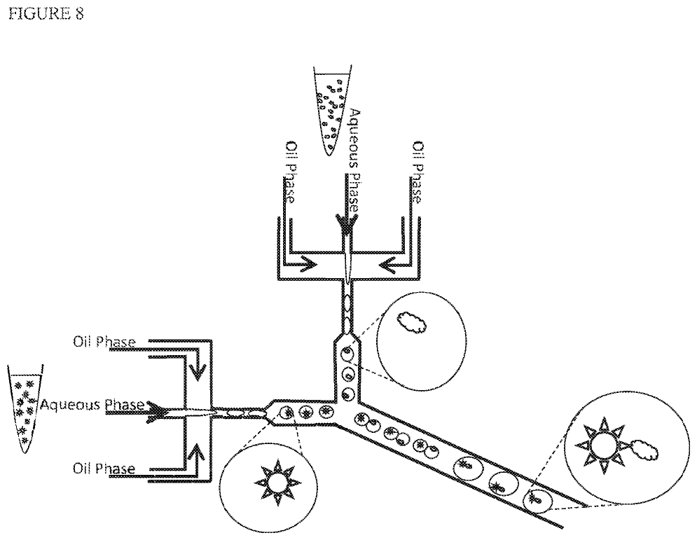

In another embodiment, the disclosed methods and material complexes can be combined with a non-miscible fluid in a controlled or engineered method to form droplets containing the material complexes; and the disclosed methods, materials, and/or biologicals can be combined with a non-miscible fluid in a controlled or engineered method to form droplets containing biologicals (FIG. 8). The resulting droplets can be controllably or un-controllably merged, so each droplet can contain material precursors, material complexes, target biologicals, or any combination thereof.

### EXAMPLES

The following Examples further illustrate the salient aspects of the invention. The Examples are provided only for illustration purposes and are not intended to necessarily indicate the optimal ways of practicing the invention or optimal results that can be obtained.

### Example 1

As an example of the experimental work, the synthesis of lactose-sand (FIG. 2-A) followed these steps: 5 grams of fine sand was rinsed with 20 ml DI water while on a medium frit filter. They were then mixed with 10 ml pH 8.5 (20 mM) borate buffer and allowed to stir for 10 minutes at room temperature. Thirty nine mg of 1,1′-carbonyldiimidazole (0.24 mmol, MW 162.15) was then added to the suspension and allowed to react for 2 hours before adding 190 mg of β-D-lactose (0.55 mmol). The resulting mixture was allowed to stir for 4 days at room temperature. The final suspension was filtered and the solid was rinsed with de-ionized (DI) water. The wetness of the solid was preserved.

### Example 2

As an example of the experimental work, the synthesis of lactose-Sepharose® (FIG. 2-B) followed these steps: 5 grams of wet Sepharose (ca. 5 wt. % in water) was mixed with 10 ml pH 8.5 (20 mM) borate buffer and allowed to stir for 10 minutes at room temperature. Thirty nine mg of 1,1′-carbonyldiimidazole (0.24 mmol, MW 162.15) was then added to the suspension and allowed to react for 2 hours before adding 190 mg of β-D-lactose (0.55 mmol). The resulting mixture was allowed to stir for 4 days at room temperature. The final suspension was filtered and the solid was rinsed with 100 ml DI water. The wetness of the solid was preserved.

### Example 3

As an example of the experimental work, the synthesis of lactose-PGMA (FIG. 2-C) followed these steps: A 100 ml single neck round bottom flask and a magnetic bar were dried under vacuum while hot. Fifty ml dry tetrahydrofuran was added followed by 1.24 g (14 mmol) of 1,4-diaminobutane. While stirring the solution, 200 mg PGMA (1.4 mmol equivalents of the repeat unit) was added. The solution was then allowed to stir at room temperature for 10 min before starting the in-situ evacuation into a cold trap, using the vacuum line. The reaction flask was gently heated using a heating gun in order to ensure the removal of all volatile reagents. To the resulting oil-like product, 50 ml DI water were added leading to the precipitation of a white film-like solid. This solid was then filtered on a medium frit and rinsed with 300 ml DI water. The yield was 0.529 g of PGMA-NH₂. The final polymer was efficiently dried and stored at low temperature.

One hundred and ten mg of the resulting intermediate, PGMA-NH₂, was mixed with 10 ml pH 8.5 20 mM borate buffer and allowed to stir for few minutes at room temperature. Nineteen mg of 1,1′-carbonyldiimidazole was then added to the suspension and allowed to stir for 1 hour before adding 0.055 g of β-D-lactose. The final mixture was allowed to stir for two days at room temperature followed by filtering through a medium frit and rinsing with 50 ml DI water. The wetness of the solid was preserved.

### Example 4

As an example of the experimental work, the synthesis of lactose-[branching]-sand (FIG. 3-A) followed these steps: Five grams of fine sand was vigorously stirred with 20 ml DI water, then filtered through a medium frit. They were then mixed with 10 ml pH 8.5 20 mM borate buffer and allowed to stir for few minutes at room temperature. Sixteen mg of 1,1′-carbonyldiimidazole (0.1 mmol, MW 162.15) was then added to the suspension and allowed to stir for 2 more hours before adding 0.25 g of Hyperbranched bis-MPA polyester-16-hydroxyl (0.1425 mmol, 2.28 mmol.eq. OH). After two additional hours, 0.37 g (2.28 mmol) of 1,1′-carbonyldiimidazole was added to the suspension and allowed to stir for 2 more hours before adding 3.9 g (11.4 mmol) of β-D-lactose. Five ml of the pH 8.5 borate buffer was then added. The final “almost clear” mixture was allowed to stir for two days at room temperature. The final solution was filtered through a medium frit and rinsed with 50 ml DI water, isolating 4.8943 g of sand complex the color of which was similar to that of the starting sand. The wetness of the solid was preserved.

### Example 5

As an example of the experimental work, the synthesis of lactose-[branching]-Sepharose (FIG. 3-B) followed these steps: One gram of wet Sepharose (ca. 5 wt. % in water) was mixed with 10 ml pH 8.5 20 mM borate buffer and allowed to stir for few minutes at room temperature. Thirty two mg of 1,1′-carbonyldiimidazole (0.2 mmol, MW 162.15) was then added to the suspension and allowed to stir for 2 more hours before adding 0.5 g of Hyperbranched bis-MPA polyester-16-hydroxyl (0.285 mmol, 4.56 mmol.eq. OH). After two additional hours, 0.74 g (4.56 mmol) of 1,1′-carbonyldiimidazole was added to the suspension and allowed to stir for 2 hours before adding 7.8 g (22.8 mmol) of β-D-lactose. Additional 5 ml of the pH 8.5 buffer was added. The final white mixture was allowed to stir for two days at room temperature. Fifty ml DI water were added to the final dense white solution to ensure dissolution of all free reagents. The final solution was filtered through a medium frit and rinsed with 50 ml DI water. The wetness of the solid was preserved.

### Example 6

As an example of the experimental work, the synthesis of lactose-[branching]-PGMA (FIG. 3-C-1), including a dendrimer, followed these steps: Hundred mg of PGMA-NH₂ (0.4 mmol equivalents of the repeat unit) was mixed with 50 ml pH 8.5 20 mM borate buffer and allowed to stir for few minutes at room temperature. Sixty four mg of 1,1′-carbonyldiimidazole (0.4 mmol, MW 162.15) was then added to the suspension and allowed to stir for 2 hours before adding 1 g of Hyperbranched bis-MPA polyester-16-hydroxyl (0.57 mmol, 9.12 mmol.eq. OH). After two additional hours, 1.48 g (9.12 mmol) of 1,1′-carbonyldiimidazole was added to the suspension and allowed to stir for 2 more hours before adding 15.6 g (45.6 mmol) of β-D-lactose. The final white mixture was allowed to stir for two days at room temperature. Fifty ml DI water was added to the final dense white solution to ensure dissolution of all free reagents. The final solution was filtered through a medium frit and rinsed with 50 ml DI water. The wetness of the solid was preserved.

### Example 7

As an example of the experimental work, the synthesis of lactose-[branching]-PGMA (FIG. 3-C-2), including chitosan, followed these steps: Four hundred ml of 0.5% acetic acid in DI water was prepared by adding 2 g of the acid to 400 mL of water. To this acid solution, 2 g of Chitosan was added and the solution was allowed to stir at room temperature for 5 min until becoming monophasic. Then, 200 mg of PGMA was added and the final suspension was allowed to stir at room temperature for two hours. The final off-white suspension was then filtered through a medium frit and the solid was washed with 100 ml of DI water. The isolated solid was re-suspended in 10 ml DI water. Its pH was ca. 4. One drop of a sodium carbonate solution (5 wt. % sodium carbonate solution prepared by dissolving 500 mg of Na₂CO₃ in 9.5 g DI water) was added to increase the pH to ca. 9. The now basic mixture was filtered and rinsed with 50 ml DI water. The yield was 140 mg of chitosan-PGMA. Hundred mg of this intermediate was suspended in 10 ml pH 8.0 borate buffer. 0.148 g (0.9 mmol) of 1,1′-carbonyldiimidazole was added to the suspension and allowed to stir for 2 hours before adding 1.56 g (4.5 mmol) of β-D-lactose. The final mixture was allowed to stir for two days at room temperature. The final solution was filtered through a medium frit, rinsed with 100 ml DI water.

### Example 8

As yet another example of the experimental work, the synthesis of lactose-[branching]-sand follows these steps: Five grams of fine sand are vigorously stirred with 20 ml DI water, then filtered through a medium frit. They are then mixed with 10 ml pH 8.5 20 mM borate buffer and allowed to stir for few minutes at room temperature. Sixteen mg of 1,1′-carbonyldiimidazole (0.1 mmol, MW 162.15) are then added to the suspension and allowed to stir for 2 more hours before adding branched poly(ethylene glycol) (2.28 m-mmol.eq. OH). After two additional hours, 0.37 g (2.28 mmol) of 1,1′-carbonyldiimidazole is added to the suspension and allowed to stir for 2 more hours before adding 3.9 g (11.4 mmol) of β-D-lactose. Five ml of the pH 8.5 borate buffer are then added. The final mixture is allowed to stir for two days at room temperature. The final solution is filtered through a medium frit and rinsed with 50 ml DI water. The wetness of the solid is preserved.

### Example 9

As yet another example of the experimental work, the synthesis of lactose-[branching]-Sepharose follows these steps: One gram of wet Sepharose (ca. 5 wt. % in water) is mixed with 10 ml pH 8.5 20 mM borate buffer and allowed to stir for few minutes at room temperature. Thirty two mg of 1,1′-carbonyldiimidazole (0.2 mmol, MW 162.15) are then added to the suspension and allowed to stir for 2 more hours before adding branched poly(ethylene glycol) (4.56 mmol.eq. OH). After two additional hours, 0.74 g (4.56 mmol) of 1,1′-carbonyldiimidazole is added to the suspension and allowed to stir for 2 hours before adding 7.8 g (22.8 mmol) of β-D-lactose. Additional 5 ml of the pH 8.5 buffer is added. The final mixture is allowed to stir for two days at room temperature. Fifty ml DI water are added to the final solution to ensure dissolution of all free reagents. The final solution is filtered through a medium frit and rinsed with 50 ml DI water. The wetness of the solid is preserved.

### Example 10

As yet another example of the experimental work, the synthesis of lactose-[branching]-PGMA, including a branched polymer, follows these steps: Hundred mg of PGMA-NH₂ (0.4 mmol equivalents of the repeat unit) are mixed with 50 ml pH 8.5 20 mM borate buffer and allowed to stir for few minutes at room temperature. Sixty four mg of 1,1′-carbonyldiimidazole (0.4 mmol, MW 162.15) are then added to the suspension and allowed to stir for 2 hours before adding branched poly(ethylene glycol) (9.12 mmol.eq. OH). After two additional hours, 1.48 g (9.12 mmol) of 1,1′-carbonyldiimidazole are added to the suspension and allowed to stir for 2 more hours before adding 15.6 g (45.6 mmol) of β-D-lactose. The final mixture is allowed to stir for two days at room temperature. Fifty ml DI water are added to the final solution to ensure dissolution of all free reagents. The final solution is filtered through a medium frit and rinsed with 50 ml DI water. The wetness of the solid is preserved.

### Example 11

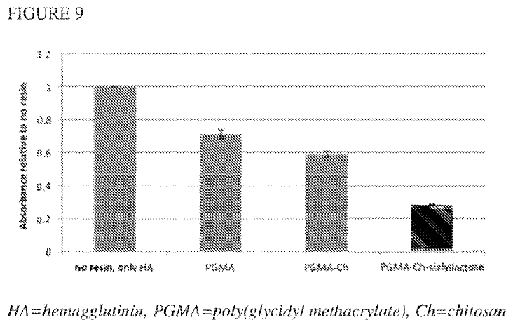

As yet another example of the experimental work, sialyllactose-complexed with PGMA was prepared. Since influenza's envelope protein, hemagglutinin (HA), is known to strongly bind to innate sialic acid in membranes of host cells, covalently attaching sialyllactose onto insoluble supports would allow virus adsorption to these surfaces. To this end, sialyllactose-complexed with PGMA was prepared following FIG. 3-C-2 using 6′-sialyllactose instead of β-D-lactose as the starting material. The linker therein was chitosan. Chemical derivatization of the material was monitored by recombinant HA binding assays (quantified by the Bradford test) (FIG. 9).

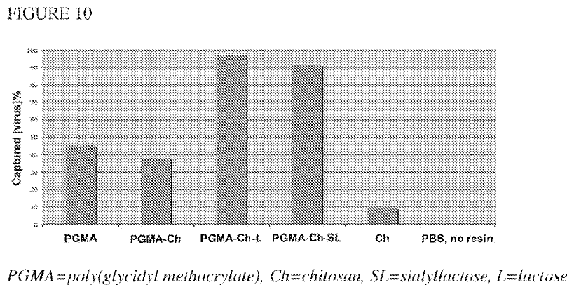

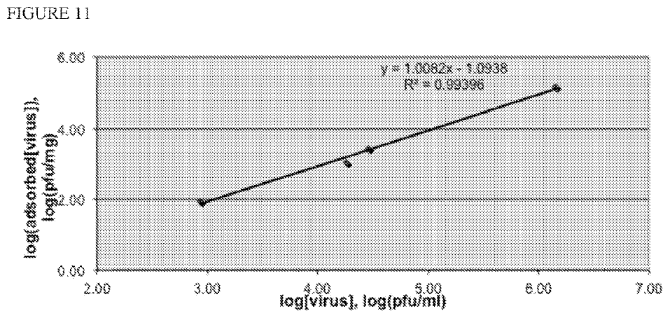

The PGMA-attached sialyllactose along with a set of controls were tested in a buffered (PBS) aqueous solution of PR8 strain of influenza-A virus, with the viral titers in the supernatants quantified using the plaque assay. The results revealed that PGMA-chitosan-lactose removed more than 98% of the virus from solution (Table 1 and FIG. 10). Furthermore, data showed that the virus adsorption to the disclosed material complexes follows a linear isotherm; the relatively constant percentage of adsorbed influenza A to the material complexes reflects Freundlich isotherm that describes adsorption of entities on suspended surfaces at very low surface coverage. Indeed, the linearity between log (adsorbed virus) and log (initial virus) was confirmed by obtaining a R² coefficient=0.994 (Table 2 and FIG. 11).

TABLE 1
Quantification of influenza A attachment to insoluble materials

| | Average # of virus | | |
|---|---|---|---|
| | in supernatant | Standard | Captured [virus]% |
| Material | (×10\hat{ }3 pfu/ml) | Deviation | compared to PBS |
| PGMA | 7.7 | 1.1 | 45 |
| PGMA-Ch | 8.7 | 2.3 | 38 |
| PGMA-Ch-L | 0.4 | 0.2 | 97 |
| PGMA-Ch-SL | 1.2 | 0.2 | 91 |
| Ch | 12.7 | 2.1 | 9 |
| PBS, no | 14 | 0.8 | 0 |
| material | | | |

PGMA = poly(glycidyl methacrylate),
Ch = chitosan,
SL = sialyllactose,
L = lactose

TABLE 2
Activity of complexed poly(glycidyl methacrylate) polymer
while varying the initial titer of influenza A

| Starting [Virus] | Adsorbed [Virus] | |
|---|---|---|
| (pfu/ml) | (pfu/mg) | % Adsorbed [virus] |
| 1,433,333 | 142133 | 99.2 |
| 28,333 | 2791 | 98.5 |
| 863 | 85 | 98.8 |
| 18000 | 1100 | 93.9 |

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

1. A method for immobilizing a biological comprising:
mixing a fluid sample comprising the biological with a material complex comprising a hydroxyl-, amino-, mercapto or epoxy-containing material that is fluid-insoluble and at least one receptor selected from lactose, lactose derivative, mono- or poly-saccharide, heparin, chitosan, deoxyribonucleic acid, ribonucleic acid, peptide, photoreceptor, or any combination thereof, wherein the receptor is bound to the material;
suspending the fluid sample in at least one immiscible fluid; and
separating the biological from the fluid sample by adsorbing the biological to the material complex.

2. The method of claim 1, wherein the biological is selected from the group consisting of cell, cell product, tissue, tissue product, blood, blood product, body fluid, product of body fluid, protein, nucleic acid, vaccine, antigen, antitoxin, biological medicine, biological treatment, virus, virus product, microorganism, microorganism product, fungus, yeast, alga, bacterium, prokaryote, eukaryote, Staphylococcus aureus, Streptococcus, Escherichia coli (E. coli), Pseudomonas aeruginosa, mycobacterium, adenovirus, rhinovirus, smallpox virus, influenza virus, herpes virus, human immunodeficiency virus (HIV), rabies, chikungunya, severe acute respiratory syndrome (SARS), polio, malaria, dengue fever, tuberculosis, meningitis, typhoid fever, yellow fever, ebola, shingella, listeria, yersinia, West Nile virus, protozoa, fungi Salmonella enterica, Candida albicans, Trichophyton mentagrophytes, poliovirus, Enterobacter aerogenes, Salmonella typhi, Klebsiella pneumonia, Aspergillus brasiliensis, methicillin resistant Staphylococcus aureus (MRSA), any derivative thereof, or any combination thereof.

3. The method of claim 1, wherein the material is selected from the group consisting of agarose, sand, textiles, metallic particles (including nanoparticles), magnetic particles (including nanoparticles), glass, fiberglass, silica, wood, fiber, plastic, rubber, ceramic, percelain, stone, marble, cement, biological polymers, natural polymers, synthetic polymers, poly acrylamide polymers, poly lactic polymers, gel, colloidal gel, hydrogel, any derivative thereof, or any combination thereof.

4. The method of claim 1, wherein the receptor is bound directly to the material.

5. The method of claim 1, wherein the receptor is bound indirectly to the material, via a linker.

6. The method of claim 5, wherein the linker is selected from the group consisting of linear poly(ethylene glycol) (PEG), branched PEG, linear poly(ethylenimine) (PEI, various ratios of primary:secondary:tertiary amine groups), branched PEI, a dendron, a dendrimer, a hyperbranched bis-MPA polyester-16-hydroxyl, chitosan, any derivative thereof, or any combination thereof.

7. The method of claim 5, wherein the inter-bonding between any combination of receptor, material, and the linker is achieved using at least one chemical coupling reagent.

8. The method of claim 7, wherein the coupling reagent is selected from the group consisting of 1,1′-carbonyldiimidazole (CDI), N,N-Dicyclohexylcarbodiimide (DCC), N-(3-Dimethylaminopropyl)-N′-ethylcarbodiimide hydrochloride (EDC or EDCI), or any combination thereof.

9. The method of claim 5, wherein the inter-bonding between any combination of receptor, material and the linker is achieved using physical attachment, or a combination of chemical and physical attachments.

10. The method of claim 9, wherein the physical attachment is achieved by deposition of the receptor, the linker, or a combination thereof, onto the material in a controlled fashion, a non-controlled fashion, or a combination thereof.

11. The method of claim 1, wherein the material is chemically functional and the chemical functionality is amino, ammonium, hydroxyl, mercapto, sulfone, sulfinic acid, sulfonic acid, thiocyanate, thione, thial, thiol, carboxyl, halocarboxy, halo, imido, anhydrido, alkenyl, alkynyl, phenyl, benzyl, carbonyl, formyl, haloformyl, carbonato, ester, alkoxy, phenoxy, hydroperoxy, peroxy, ether, glycidyl, epoxy, hemiacetal, hemiketal, acetal, ketal, orthoester, orthocarbonate ester, amido, imino, imido, azido, azo, cyano, nitrato, nitrilo, nitrito, nitro, nitroso, pyridinyl, phosphinyl, phosphonic acid, phosphate, phosphoester, phosphodiester, boronic acid, boronic ester, borinic acid, borinic ester, any derivative thereof, or any combination thereof.

12. The method of claim 11, wherein the epoxy-containing material is Poly(glycidyl methacrylate) (PGMA).

13. The method of claim 11, wherein the amino-containing material is PGMA-NH₂.

14. The method of claim 11, wherein the hydroxyl, mercapto, or amino group is formed on a surface of the material by modifying the substrate by a chemical transformation.

15. The method of claim 14, wherein the chemical transformation comprising a hydrolysis reaction with an acid, a base, or a combination thereof.

16. The method of claim 1, wherein the material complex is formed within the fluid sample, and wherein the biological is encapsulated or immoblized in or on the material complex.

17. The method of claim 1, further comprising separating the immobilized biological from the fluid sample by filtration, decantation, applying gravity or magnetic forces, flow cytometry, fluorescence-activated cell sorter, or any combination thereof.

18. The method of claim 1, further comprising releasing the immobilized biological from the material complexe.

19. The method of claim 18, wherein the immobilized biological is released from the material complex by light-inducing variations, enzymatic activity, physical variations, chemical variations, or any combination thereof.

20-27. (canceled)

28. A material complex comprising:
a hydroxyl-, amino-, mercapto or epoxy-containing material and at least one receptor bound to the material and selected from lactose, lactose derivative, mono- or poly-saccharide, heparin, chitosan, deoxyribonucleic acid, ribonucleic acid, peptide, photoreceptor, or any combination thereof,
wherein the material complex is dispersed in a first fluid, and
wherein the first fluid is suspended in an immiscible second fluid.
